(19) United States
(12) Patent Application Publication
(10) Pub. No.: US 2005/0214471 A1
(43) Pub. Date: Sep. 29, 2005

(54) Molecular templating of a surface

(76) Inventors: James Theobald, Nottingham (GB); Neil S. Oxtoby, Nottingham (GB); Peter Beton, Nottingham (GB); Neil Champness, Nottingham (GB)

Correspondence Address: MATHEWS, SHEPHERD, MCKAY, & BRUNEAU, P.A., 100 THANET CIRCLE, SUITE 306, PRINCETON, NJ, 08540, US

(21) Appl. No.: 11/025,257

(22) Filed: Dec. 29, 2004

Related U.S. Application Data
(60) Provisional application No. 60/533,827, filed on Dec. 31, 2003.

(51) Int. Cl. B05D 1/36; B05D 7/00; C12Q 1/68; G03F 9/00

(52) U.S. Cl. 427/402; 430/5; 430/4; 427/407.1; 977/DIG. 1

(57) ABSTRACT

The present invention describes a method for producing a large area two-dimensional nanoscale network on the surface of a substrate. The network is formed by depositing a sub-mono-layer of molecule A onto the surface of the substrate followed by a different molecule B. The formation of the network relies on the hetero-molecular hydrogen bonding between molecules A and B to be stronger than the homo-molecular hydrogen bonding. By appropriate choice of molecules A and B, together with the substrate, it is possible to manipulate and control the structure, dimensions and chemical functionality of the network. The pores of the network can act as containment vessels for other molecules and be made sufficiently large to accommodate several large molecules or atomic/molecular clusters or particles.

### FIELD OF THE INVENTION

This invention refers to a method of producing and structuring molecular networks on a substrate. More particularly, but not exclusively, the invention relates to a method of producing and structuring nanoscale molecular networks on a substrate. These networks can then be used either as a location mechanism for large molecules or as a mode of forming or transferring nanoscale patterns on to a surface.

### BACKGROUND OF THE INVENTION

It is highly desirable to be able to reproducibly deposit a controllable nanoscale pattern onto a surface. Such a pattern can then be used as either a containment vessel or as a means of surface lithography. Technological applications include high-speed computing, high density storage and display, and optical communications through devices such as the single-electron transistor and quantum dot laser.

Non-covalent directional interactions between different molecules provide a pre-determined recognition pathway which has been widely exploited in solution-based supramolecular chemistry to form functional nanostructures such as capsules, switches and prototype machines (for example Lehn, 2000).

Recently there have been major advances in transferring the protocols of supramolecular organisation to two dimensional surface based assembly (Hecht, 2003; de Feyter et al., 2003). Several groups have demonstrated structures which may be stabilised by hydrogen bonding (for example Barth et al., 2000), dipolar coupling (for example Yokoyama et al., 2001) or metal co-ordination (Lin et al., 2002). These include isolated rows (for example Barth et al., 2000), clusters (for example Furukawa et al., 2000) and networks (for example Berner et al., 2001) as well as more complex multi-component arrangements (de Wild et al., 2002).

The ability to create networks capable of accommodating a single fullerene molecule within their pores has been demonstrated previously (Gimzewski et al., 1997; Cuberes et al., 1997). However, accommodating only one molecule limits the scope of possible applications based on this approach to the positioning of isolated non-interacting molecules. Therefore, it is desirable to form a self-assembled surface network containing pores that are sufficiently large to accommodate several large molecules or nano-scale particles. Such a fundamental and major advance would give rise to many exciting technological and scientific opportunities.

The creation of such networks is possible using the deposition method described in patent application WO 02/086200, which concerns a deposition method for forming molecular and atomic patterns on a substrate. However, the control over the arrangement of the molecules is poor and determined by packing density. Instead, a controllable and reproducible method is required whereby the structure, dimensions and chemical functionality of the network is determined by the choice of molecules used to form the network, and the substrate on which the network is formed.

### SUMMARY OF THE INVENTION

The present invention describes a method for producing a large area two-dimensional nanoscale network on the surface of a substrate. The network is formed by depositing a sub-mono-layer of molecule A onto the surface of the substrate followed by a different molecule B. The formation of the network relies on the hetero-molecular hydrogen bonding between molecules A and B to be stronger than the homo-molecular hydrogen bonding. Thus, by appropriate choice of molecules A and B, together with the substrate, it is possible to manipulate and control the structure, dimensions and chemical functionality of the network. The pores of the network can act as containment vessels for other molecules, atoms and nano particles that can be held non-specifically by Van der Waals forces or via chemical interactions/bonds which can be made to be specific for a chosen molecule. The pores can be made sufficiently large to accommodate several large molecules or atomic/molecular clusters or particles. Alternatively, the network can be used as a lithographic tool to form or transfer nanoscale patterns and structures on to a surface.

According to a first aspect of the present invention there is provided a method of producing and/or structuring a molecular network upon a substrate comprising the steps of:
depositing a first sub-layer comprising a first molecular species upon a surface;
depositing a second sub-layer comprising a second molecular species upon the surface, the first and second molecular species being different molecular species;
bonding at least at portions of the first molecular species to at least a portion of the second molecular species so as to form a molecular network.

Either, or both, of the first or, and, sub-layers may comprise sub-monolayer coverages of the first or, and, second molecular species respectively. Step (iii) may comprise hetero-molecular bonding between the first and second molecular species, typically hetero-molecular hydrogen bonding.

The method may include varying the structure and/or dimensions of the network by varying either, or both, of the first or, and, second molecular species. The method may include varying the chemical functionality, or/and the chemical selectivity, of the network by varying either, or both, of the first or, and, second molecular species.

The method may include retaining any one, or combination, of the following within at least one pore of the network: a molecule, typically not of the either of the first or second molecular species, an atom, a nano particle, one or more large, possibly macro, molecules, atomic cluster, molecular cluster. The method may include using non-specific Van der Waals interactions, or specific chemical interactions, or a combination of both, to retain any one, or combination, of the following within at least one pore of the network: a molecule, typically not of the either of the first or second molecular species, an atom, a nano particle, one or more large, possibly macro, molecules, atomic cluster, molecular cluster; within at least one pore of the network.

According to a second aspect of the present invention there is provided a photolithographic mask, or reticle, fabricated using the method of the first aspect of the present invention.

According to a third aspect of the present invention there is provided an electronic, optoelectronic or photonic circuit fabricated using the mask, or reticle, of the second aspect of the present invention.

According to a fourth aspect of the present invention there is provided a data storage medium fabricated using the method of the first aspect of the present invention.

According to a fifth aspect of the present invention there is provided a display device according fabricated using the method of the first aspect of the present invention.

### BRIEF DESCRIPTION OF THE DRAWINGS

In the following the invention will be described, by way of example only, with reference to the preferred embodiments illustrated in the accompanying drawings, in which:

FIG. 1a is a schematic drawing of perylene tetra-carboxylic di-imide (PTCDI) molecule;

FIG. 1b is a schematic drawing of melamine molecule;

FIG. 1c is a schematic drawing of network formed by a melamine molecule and three PTCDI molecules;

FIG. 2 shows examples of other possible molecules suitable for network formation;

FIG. 3a shows an STM image of PCTDI-melamine network. Scale bars, 3 nm;

FIG. 3b is a schematic diagram showing the registry of the network with the surface;

FIG. 4a shows an STM image of C60 heptamers trapped within nano-scale vessels of a Melamine-PTCDI network. Scale bar, 5 nm;

FIG. 4b is a schematic diagram of C60 heptamer trapped within Melamine-PTCDI network;

FIG. 5a shows an STM image showing the Melamine-PTCDI network, C60 heptamers and the raised C60 honeycomb network. Scale bar, 5 nm; and

FIG. 5b shows an STM image of a low defect termination of a melamine-PTCDI network with C60 (Scale bar, 10 nm).

### DESCRIPTION OF THE PREFERRED EMBODIMENTS

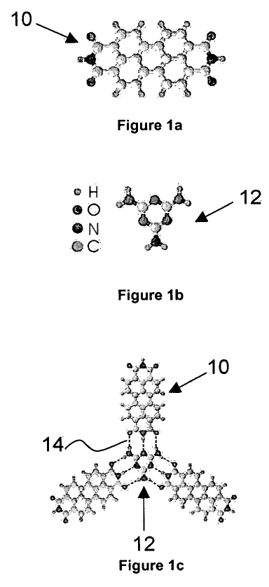

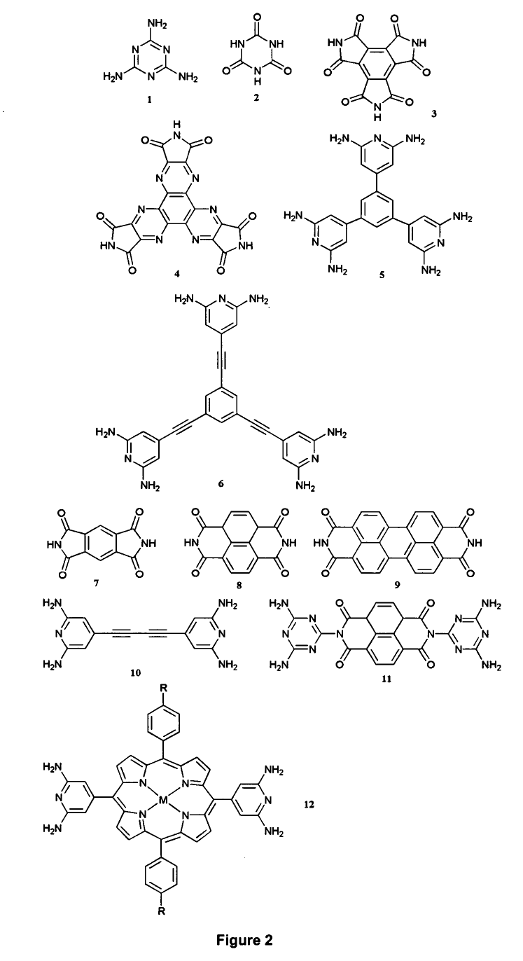

Molecular entrapment in nanoscale vessels formed by surface supramolecular assembly, the method of self-assembly of a nano-scale network described here is a bimolecular method that requires the two molecules, A and B, to exhibit stronger hetero-molecular hydrogen bonding compared to homo-molecular hydrogen bonding, and also to have a compatible molecular geometry. Perylene tetra-carboxylic di-imide (PTCDI) 10, illustrated in FIG. 1a, and melamine 12, illustrated in FIG. 1b, are two such molecules that exhibit these properties. FIG. 1c illustrates the compatibility of the molecular geometries of melamine 12 and PTCDI 10 which results in three hydrogen bonds 14 per melamine-PTCDI pair. It is understood that melamine 12 and PTCDI 10 are used in the following description to exemplify the invention only and other molecular pairs that exhibit the similar properties of compatible molecular geometry and strong hetero-molecular hydrogen bonding compared to homo-molecular hydrogen bonding may also be used. Examples of further molecules that can be used are shown in FIG. 2.

The formation of the nano-scale network is a two stage process where a sub-monolayer of the molecule A is first deposited onto a prepared substrate. Molecule B is then deposited on the substrate and the network is formed. Methods of deposition that may be used to carry this out include, but are not limited to, ultra-high vacuum deposition and solution based deposition.

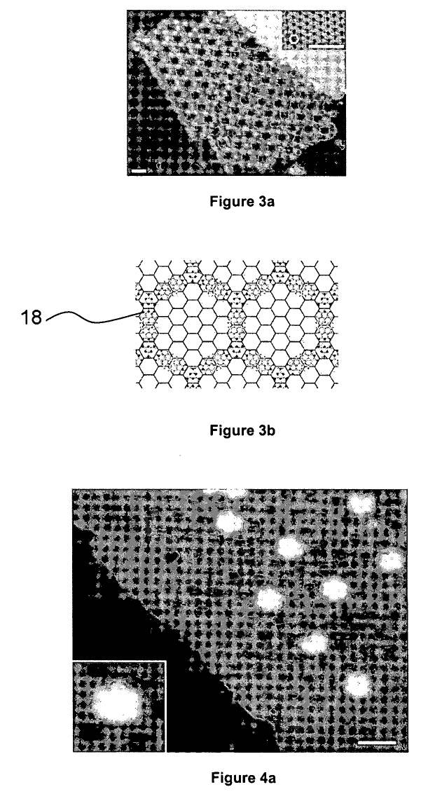

The melamine-PTCDI network illustrated in FIG. 3 was prepared under ultra-high vacuum conditions (base pressure ˜5×10⁻¹¹ Torr). PTCDI 10 and melamine 12 were placed in effusion cells and sublimed through heating to ˜360° C. and ˜100° C. respectively, onto a Ag/Si(111)-√3×√3R30° surface held at room temperature. The method of deposition and preparation of such substrates is well-known to those skilled in the art.

The first step in the formation of the network 18 was the deposition of 0.1-0.3 mono-layers of PTCDI 10 to form close packed islands and short chains on the surface of the substrate. Melamine 12 was then deposited while the sample was annealed at ˜100° C. The annealing provides sufficient thermal energy for molecules to detach from PTCDI islands and diffuse across the surface. These PTCDI molecules 10 interact with melamine 12 to nucleate the hexagonal network 18 which then grows through further capture of diffusing molecules.

STM images of the resulting melamine-PTCDI network 18 are shown in FIG. 3a. The network has principal axes at 30° to those of the Ag/Si(111)-√3×√3R30° surface and a lattice constant 3√3aₒ=34.6 Å. The geometry and dimensions of the nano-scale network formed by the bi-molecular pair is determined by geometries and dimensions of the two molecules used in the network's formation. The hexagonal structure seen with the melamine-PTCDI network 18 is determined by the threefold symmetry of the melamine 12 which forms the vertices of the network while the straight edges correspond to PTCDI 10. Alternative geometries such as rectangles, wires and triangles are achievable through appropriate choice of molecules.

The use of bimolecular assembly using long molecules to define the edges of the network results in pores which are much larger than the constituent building blocks of the networks and enables their use as traps, or vessels, which may be used to co-locate several large molecules, clusters or particles. This potential is demonstrated through the sublimation of C₆₀ 20 onto the hexagonal melamine-PTCDI network 18 top form a new fullerene nanophase—the heptamer 22—in the pores. However, sublimation and other methods may be used to fill the pores with other technologically exciting molecular species or combination of species, clusters or particles.

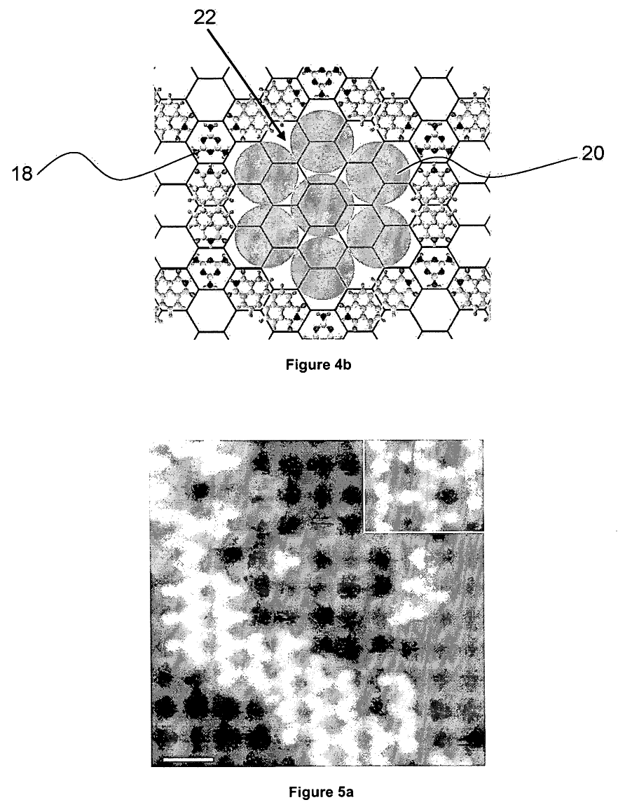

FIG. 4a shows an STM image acquired following the deposition of 0.03 monolayers (ML) of C₆₀ 20. Heptameric clusters 22 of C₆₀ 20, in which molecules are ordered in a compact hexagonal arrangement are seen to have formed within the pores. The clusters 22 formed in different pores are aligned, and are all oriented parallel to the principal axes of the Si(111) surface. The molecular arrangement of the heptamers 22 has been deduced from the STM images of FIG. 4a and are shown in FIG. 4b. Clusters of fewer molecules are also observed. For example there are clusters of six molecules in FIG. 4a and clusters of 2-5 molecules have also been observed, while many pores remain empty for this coverage of C₆₀ 20.

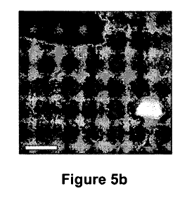

As the coverage of C₆₀ 20 is increased the fraction of pores capturing adsorbed molecules and stabilising heptameric clusters 22 increases. This is accompanied by the adsorption of C₆₀ 20 directly above the PTCDI 10 and melamine 12 molecules reproducing the underlying hexagonal network 18. STM images showing second layer C₆₀ 20, heptamers 22 and the melamine-PTCDI network 18 in close proximity are shown in FIG. 5a.

A further increase in C₆₀ 20 coverage results in a near perfect termination of the second layer as shown in STM images FIG. 5b. An array of C₆₀ 20 molecules sits directly above the melamine-PTCDI network 18 and the lateral positions within this array correspond exactly to those of a hexagonally close packed layer. However, the elevation of the hexagonal network's 18 constituent molecules results in an increase in their separation with molecules at the heptamer 22 edge and this arrangement thus constitutes a new surface phase of fullerene which is controlled and templated by the hydrogen bonded network.

Further deposition of C₆₀ 20 up to a total of 3 ML does not lead to the formation of higher layers of fullerene on the hexagonal network. This observation is attributed to the absence of sites in the termination shown in FIG. 5 which are suitable for the stable nucleation of higher layers.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents which may be resorted to can be considered to fall within the scope of the invention.

### REFERENCES

de Wild, M., Berner, S., Suzuki, H., Yanagi, H., Schlettwein, D., Ivan, S., Baratoff, A., Guentherodt H-J. & Jung, T. A. A novel route to molecular self-assembly: Self-intermixed monolayer phases. Chem. Phys. Chem. 3, 881-885 (2002).
Balzani, V., Credi, A., Raymo, F. M. & Stoddart, J. F. Artificial molecular machines. Angew. Chem. Int. Ed. 39, 3349-3391 (2000).
Barth, J. V., Weckesser, J., Cai., C., Gunter, P., Burgi, L., Jeandupeux, O. & Kern, K. Building supramolecular nanostructures at surfaces by hydrogen bonding. Angew. Chem. Int. Ed. 39, 1230-1234 (2000).
Berner, S., Brunner, M., Ramoino, L., Suzuki, H., Guentherodt, H-J. & Jung, T. A., Time evolution analysis of a 2D solid-gas equilibrium: a model system for molecular adsorption and diffusion. Chem.Phys.Lett. 348, 175-181 (2001).
Bohringer, M., Morgenstern, K., Schneider, W. D. & Berndt, R. Separation of a racemic mixture of two-dimensional molecular clusters by scanning tunneling microscopy. Angew. Chem. Int. Ed. 38, 821-823 (1999).
Chen, Q., Frankel, D. J. & Richardson, N. V. Self-assembly of adenine on Cu(110) surfaces. Langmuir 18, 3219-3225 (2002).
Cuberes, M. T., Schlittler, R. R. & Gimzewski, J. K. Room temperature supramolecular repositioning at molecular interfaces using a scanning tunneling microscope. Surf. Sci. 371, L231-L234 (1997).
De Feyter, S. & De Schryver, F. C. Two-dimensional supramolecular self-assembly probed by scanning tunnelling microscopy. Chem. Soc. Rev. 32, 139-150 (2003).
Dmitriev, A., Lin, N., Weckesser, J., Barth, J. V. & Kern, K. Supramolecular assemblies of trimesic acid on a Cu(100) surface. J. Phys. Chem. B 106, 6907-6912 (2002).
Fujita, M., Fujita, N., Ogura, K. & Yamaguchi, K. Spontaneous assembly of ten components into two interlocked, identical coordination cages. Nature 400, 52-55 (1999).
Furukawa, M., Tanaka, H. & Kawai, T. Formation mechanism of low-dimensional superstructure of adenine molecules and its control by chemical modification: a low-temperature scanning tunneling microscopy study. Surf. Sci. 445, 1-10 (2000).
Gimzewski, J. K., Jung, T. A., Cuberes, M. T. & Schlittler, R. R. Scanning tunneling microscopy of individual molecules: beyond imaging. Surf. Sci. 386, 101-114 (1997).
Griessl, S., Lackinger, M., Edelwirth, M., Hietschold, M. & Heckl, W. M. Self-assembled two-dimensional molecular host-guest architectures from trimesic acid. Single Mol. 3, 25-31 (2002).
Hecht, S. Welding, organizing, and planting organic molecules on substrate surfaces—Promising approaches towards nanoarchitectonics from the bottom up. Angew. Chem. Int. Ed. 42, 24-26 (2003).
Keeling, D. L., Oxtoby, N. S., Wilson, C., Humphry, M. J., Champness, N. R. & Beton, P. H. Assembly and processing of hydrogen bond induced supramolecular nanostructures. Nano Lett. 3, 9-12 (2003).
Lehn, J. M. Toward complex matter: Supramolecular chemistry and self-organization. Proc. Natl. Acad. Sci. (USA) 99, 4763-4768 (2002).
Lin, N., Dmitriev, A., Weckesser, J., Barth, J. V. & Kern, K. Real-time single-molecule imaging of the formation and dynamics of coordination compounds. Angew. Chem. Int. Ed. 41, 4779-4783 (2002).
Reinhoudt, D. N. & Crego-Calama, M. Synthesis beyond the molecule. Science 295, 2403-2407 (2002).
Seeman, N. C. DNA in a material world. Nature 421, 427-431 (2003).
Yokoyama, T., Yokoyama, S., Kamikado, T., Okuno, Y. & Mashiko, S. Selective assembly on a surface of supramolecular aggregates with controlled size and shape. Nature 413, 619-621 (2001).

1. A method of producing and/or structuring a molecular network upon a substrate comprising the steps of:
a) depositing a first sub-layer comprising a first molecular species on a surface of the substrate;
b) depositing a second sub-layer comprising a second molecular species on the surface of the substrate, the first and second molecular species being different molecular species;
c) bonding at least a portion of the first molecular species to at least a portion of the second molecular species so as to form a molecular network.

2. The method of claim 1 comprising depositing at least one of the first and second molecular species under ultra-high vacuum conditions.

3. The method of claim 1 comprising depositing at least one of the first and second molecular species using solution based deposition.

4. The method of claim 1 wherein the bonding between the first and second molecular species is hetero-molecular bonding.

5. The method of claim 4 wherein the hetero-molecular bonding is hetero-molecular hydrogen bonding.

6. The method of claim 5 wherein the first molecular species and the second molecular species exhibit stronger hetero-molecular hydrogen bonding compared to homo-molecular bonding.

7. The method of claim 1 where energy is provided during the deposition of the second molecular species to enable the first molecular species to diffuse across the surface such that at least a portion of the first molecular species is arranged to be able to bond with at least a portion of the second molecular species and thereby form the molecular network.

8. The method of claim 2 where energy is provided during the deposition of the second molecular species to enable the first molecular species to diffuse across the surface such that at least a portion of the first molecular species is arranged to be able to bond with at least a portion of the second molecular species and thereby form the molecular network.

9. The method of claim 3 where energy is provided during the deposition of the second molecular species to enable the first molecular species to diffuse across the surface such that at least a portion of the first molecular species is arranged to be able to bond with at least a portion of the second molecular species and thereby form the molecular network.

10. The method of claim 1 wherein the substrate is annealed during the deposition of the second sub-layer.

11. The method of claim 2 wherein the substrate is annealed during the deposition of the second sub-layer.

12. The method of claim 3 wherein the substrate is annealed during the deposition of the second sub-layer.

13. The method of claim 1 wherein at least one of the first and second molecular species is an oligo-dimide.

14. The method of claim 13 wherein the molecular species is chosen from the list comprising:
a) 1,2,4,5-Benzenetetracarboxylic 1,2:4,5-diimide
b) 1,4,5,8-Naphthalenetetracarboxylic 1,8:4,5-diimide
c) 3,4,9,10-Perylenetetracarboxylic 3,4:9,10-diimide
d) (4,4′-Di(3,5-diamino-pyridyl)butadiyne
e) 1,4,5,8-Naphthalenetetracarboxylic 1,8:4,5-diimide, N,N′-di-3,5-diamino-2,4,6-triazinyl-diimide
f) 5,15-Bis (3,5-diamino-4-pyridyl)-10,20-diphenylporphyrin derivatives.

15. The method of claim 1 wherein at least one of the first and second molecular species is an oligo-diaminopyridine.

16. The method of claim 15 wherein the molecular species is chosen from the list comprising:
a) melamine
b) cyanuric acid
c) benzo-tripyrrole-hexone
d) hexaazatriphenylene-tripyrrole-hexone
e) 1,3,5-Tris (3,5-diamino-4-pyridyl)benzene
f) 1,3,5-Tris(3,5-diamino-4-pyridylethynyl)benzene

17. The method of claim 1 comprising preparing the substrate prior to deposition of at least one of the first and second molecular species

18. The method of claim 1 wherein the substrate surface is a Ag/Si(111)-√3×√3R30° surface.

19. The method of claim 17 wherein the substrate surface is a Ag/Si(111)-√3×√3R30° surface.

20. The method of claim 1 wherein at least one of: the structure, dimensions, chemical functionality of the network formed; is determined by at least one of: the geometry and dimensions of the first and second molecular species, the substrate material, crystallographic surface.

21. The method according to claim 1 wherein the molecular network formed has at least one pore having one of the following cross-sections: hexagonal, rectangular, triangular.

22. The method of claim 1 characterised in that the network formed comprises at least one wire.

23. The method of claim 1 wherein at least one pore of the molecular network is arranged to act as a containment vessel for at least one particle.

24. The method of claim 23 wherein the particle is chosen from the list comprising: macromolecule, molecule, atom, nano-particle, at least one large molecule.

25. The method of claim 23 wherein a plurality of particles are located in the same pore.

26. The method of claim 24 wherein a plurality of particles are located in the same pore.

27. The method of claim 20 wherein the plurality particles are of the same species.

28. The method of claim 25 wherein at least one particle is a C60 molecule.

29. The method of claim 28 wherein seven C60 molecules are collocated in a containment vessel to form a heptamer.

30. The method of claim 25 wherein the plurality of particles are of at least two different species.

31. The method of claim 23 wherein the at least one particle is arranged to be held in the containment vessel using van der Waals forces.

32. The method of claim 23 wherein the at least one particle is arranged to be held in the containment vessel through chemical bonding.

33. The method of claim 23 wherein the at least one particle is arranged to be held in the containment vessel through a combination of van der Waals forces and chemical bonding.

34. The method of to claim 23 wherein at least one particle is arranged to create a second layer above the molecular network.

35. The method of claim 34 wherein the arrangement of the at least one particle in the second layer is determined by an underlying layer and constitutes a new surface phase which is templated by the network.

36. The method of claim 23 wherein the at least one particle is deposited using any suitable deposition method.

37. The method of claim 36 wherein the at least one particle is deposited using sublimation.

38. The method of claim 1 wherein the molecular network is used as a lithography mask.

39. The method of claim 38 where the lithography mask is a photolithography mask.

40. A photolithography mask fabricated using a molecular network produced using the method of claim 1.

41. A photolithography mask fabricated using a molecular network produced using the method of claim 24.

42. A circuit fabricated using a molecular network produced using the method of claim 1 wherein the circuit is of a type selected from the following list: electronic, optoelectronic, photonic.

43. A circuit fabricated using a molecular network produced using the method of claim 24 wherein the circuit is of a type selected from the following list: electronic, optoelectronic, photonic.

44. A circuit fabricated using a molecular network produced using the method of claim 40 wherein the circuit is of a type selected from the following list: electronic, optoelectronic, photonic.

45. A circuit fabricated using a molecular network produced using the method of claim 41 wherein the circuit is of a type selected from the following list: electronic, optoelectronic, photonic.

46. A display device fabricated using a molecular network produced using the method of claim 1.

47. A display device fabricated using a molecular network produced using the method of claim 24.

48. A display device fabricated using a molecular network produced using the method of claim 40.

49. A display device fabricated using a molecular network produced using the method of claim 41.

50. A data storage device fabricated using a molecular network produced using the method of claim 1.

51. A data storage device fabricated using a molecular network produced using the method of claim 24.

52. A data storage device fabricated using a molecular network produced using the method of claim 40.

53. A data storage device fabricated using a molecular network produced using the method of claim 41.

54. The method of claim 18 wherein the at least one pore has a width of 100 nm or less.

55. The method of claim 1 wherein the molecular network is self-assembled.
